(19) United States
(12) Patent Application Publication
(10) Pub. No.: US 2021/0364179 A1
(43) Pub. Date: Nov. 25, 2021

(54) MOBILE MEDICAL TESTING STRUCTURE

(71) Applicant: Holt Construction Corporation, Pearl River, NY (US)

(72) Inventor: Robert Kipp, Rockville Centre, NY (US)

(21) Appl. No.: 17/326,209

(22) Filed: May 20, 2021

Related U.S. Application Data
(60) Provisional application No. 63/027,588, filed on May 20, 2020.

(51) Int. Cl. F24F 8/95; A61L 2/10; E04H 3/08; F24F 8/10; F24F 11/89; E04H 1/12

(52) U.S. Cl. CPC F24F 8/95; A61L 2/10; E04H 3/08; F24F 8/10; F24F 2110/40; E04H 1/1277; A61L 2202/25; F24F 2203/00; F24F 11/89

(57) ABSTRACT

One or more embodiments of a device or structure for medical testing. The structure may include a shell defining an interior of the structure, at least one workstation in the interior of the structure, at least one window in the shell, each of the at least one workstation including one of the at least one window communicating between the respective workstation and an exterior of the structure, and an air control system configured to filter air and control air pressure in the interior of the structure.

### CROSS REFERENCE TO RELATED APPLICATIONS

This application claims priority to Provisional Application 63/027,588, filed May 20, 2020, which is incorporated in its entirety.

### FIELD OF THE DISCLOSURE

The present invention relates to devices or structures for medical testing; especially structures for drive through medical testing.

### BACKGROUND

During times of large-scale infection or contamination, mobile testing centers are often erected by government and health organizations. Traditionally, large tents and the like are used to house the healthcare workers. These tents are generally not secure and do not provide adequate protection for the workers (from the elements, from the infections or contaminants for which they are testing). In particular, there is great risk to workers from vehicles when drive through testing centers are implemented. The workers essentially have no physical barrier between them and the vehicles, and little to keep the vehicles at a safe distance from the workers. Similarly, there is little structural and mechanical protection for the workers from airborne pathogens and contaminants.

In addition, sometimes mobile clinics are set up. These mobile clinics generally require the patients to enter a trailer, tent, or other enclosed area where the workers provide testing services. The close proximity of the patients with the workers in a confined area can cause the workers to be at risk of transmission of infection or contaminants that are either airborne or on surfaces. Accordingly, neither of these methods are adequate for ensuring the safety of workers while conducting mass testing of patients.

### SUMMARY

One or more embodiments are provided below for a device or structure for medical testing. The structure may include a shell defining an interior of the structure, at least one workstation in the interior of the structure, at least one window in the shell, each of the at least one workstation including one of the at least one window communicating between the respective workstation and an exterior of the structure, and an air control system configured to filter air and control air pressure in the interior of the structure.

The structure may provide significant advantages over the devices known in the art. The structure may provide significant advantages in cost and safety to workers over known structures or devices. The higher air pressure in the structure caused by the air control system may keep airborne pathogens out of the interior of the structure even when the windows are open. Having higher pressure in the general-purpose room compared to the air pressure in the workstations also helps to prevent any contamination in the workstations from contaminating the other general purpose room or the other workstation(s).

Other advantageous features as well as other aspects and advantages of the invention will be apparent from the following description and the appended claims.

### BRIEF DESCRIPTION OF THE DRAWINGS

Embodiments of the present disclosure are described in detail below with reference to the following drawings. These and other features, aspects, and advantages of the present disclosure will become better understood with regard to the following description, appended claims, and accompanying drawings. The drawings described herein are for illustrative purposes only of selected embodiments and not all possible implementations and are not intended to limit the scope of the present disclosure.

FIG. 1 shows an example top view of a mobile testing center.

FIG. 2 shows an example side view of a mobile testing station.

FIG. 3 shows an example back view of a mobile testing station.

FIG. 4 shows an example top view of an interior of a mobile testing station.

FIG. 5 shows an example front view of a mobile testing station.

FIG. 6 shows workspace walls and interior doors with a small portal included in the doors.

FIG. 7 shows workspace walls and interior doors with a small portal included in the workspace walls.

### DETAILED DESCRIPTION

In the Summary above and in this Detailed Description, the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, among others, are optionally present. For example, an article “comprising” (or “which comprises”) components A, B, and C can consist of (i.e., contain only) components A, B, and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm and upper limit is 100 mm.

Certain terminology and derivations thereof may be used in the following description for convenience in reference only and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

The term “coupled to” as used herein may mean a direct or indirect connection via one or more components.

Referring now to the drawings and the following written description of the present invention, it will be readily understood by those persons skilled in the art that the present invention is susceptible to broad utility and application. Many embodiments and adaptations of the present invention other than those herein described, as well as many variations, modifications, and equivalent arrangements will be apparent from or reasonably suggested by the present invention and the detailed description thereof, without departing from the substance or scope of the present invention. This disclosure is only illustrative and exemplary of the present invention and is made merely for purposes of providing a full and enabling disclosure of the invention.

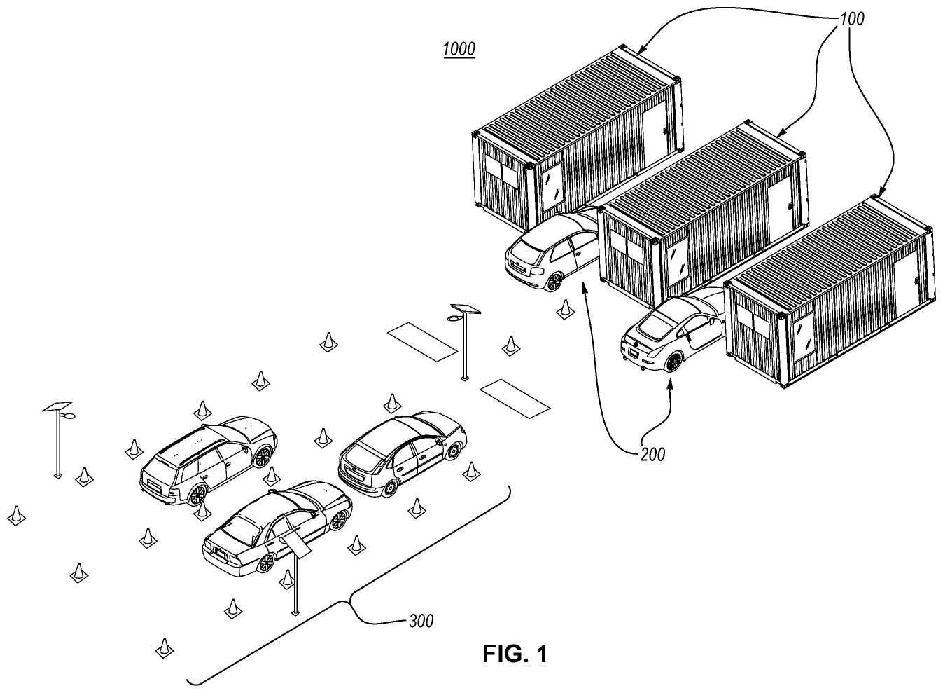

FIG. 1 shows an example top view of a mobile testing center 1000. The mobile testing center 1000 may include mobile testing structures 100 serving patients in vehicles 200 and a waiting area 300 or queue. The mobile testing center 100 may be set up in one of several different configurations. The setup shown in FIG. 1 is a non-limiting example of one setup of the mobile testing center.

As shown in FIG. 1 the mobile testing center 1000 may include multiple mobile testing structures 100. As will be shown in greater detail below, each mobile testing structure may include windows on either side of the mobile testing structure 100. Accordingly, the mobile testing center may serve patients on either side of the mobile test structure 100. As shown in FIG. 1, the mobile test structures 100 may be set up so that one worker in a first mobile test structure 100 may serve a patient in the driver seat of a vehicle, while another worker in a second mobile test structure 100 serves a patient in the passenger seat of the vehicle. In other example embodiments, traffic may be directed in a first direction on one side of a mobile test structure 100 and may be directed in a second direction on a second side of the mobile test structure 100 such that each worker in the mobile test structure 100 serves the driver side of the vehicle. In some example embodiments, traffic lights may be mounted on an exterior side of the mobile test structures 100.

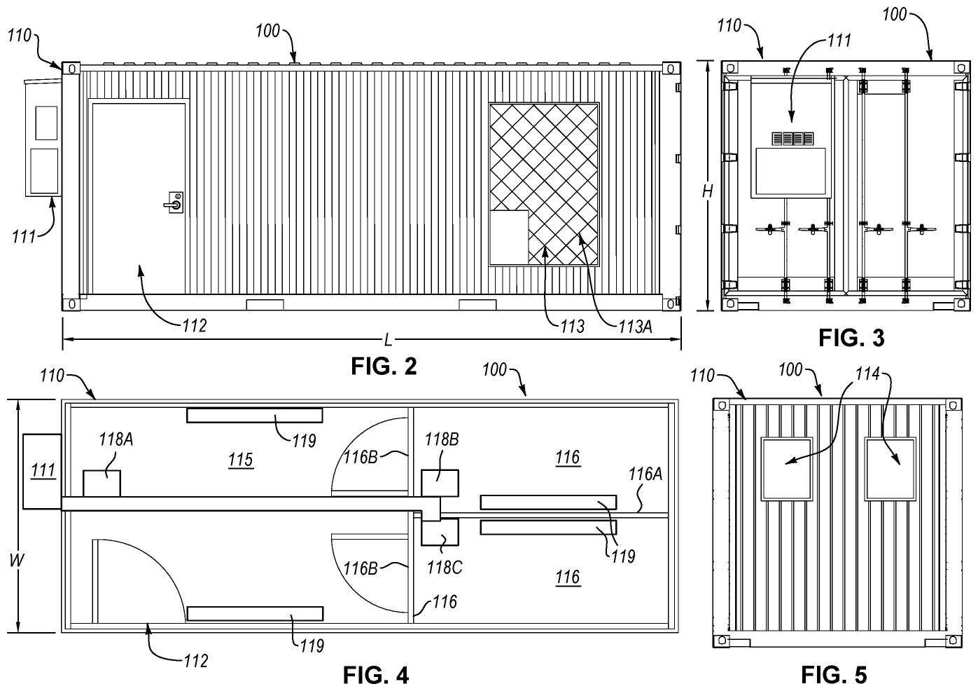

FIG. 2 shows an example side view of a mobile testing structure 100. The mobile testing structure 100 may include a shell 110, an air control system 111, an exterior door 112 and a window 113 with screen 113A. The shell 110 may be made from a shipping container or have a similar structure with metal sides, top, and bottom. ther durable materials such as wood, plastic, or composite materials may also be used to make the shell 110. The shell 110 may define an interior of the mobile testing structure 100 (i.e., what is inside the shell 110 is in the interior of the mobile testing structure). The air control system 111 may include an air filter, an air pump, an air conditioner, a heater, or any other features to help control the air flow into the mobile testing structure 100. The air control system 111 may be configured to filter and pump in air from the exterior of the mobile testing structure 100 to the interior of the mobile testing structure 100 and may be configured to control the air pressure within the general purpose room 115 and the workstations 116 by controlling how much air is pumped into the general purpose room 115 and the workstations 116. The exterior door 112 may open to allow workers access to the interior of the mobile testing structure 100 and shut to better maintain the controlled air in the interior of the mobile testing structure 100.

The service window 113 may communicate through the shell 110 between a workstation 116 and an exterior of the mobile testing structure 100 to allow a worker to serve a patient outside of the mobile testing structure 100. For example, the service window 113 may allow a worker to serve a patient in a vehicle outside the window without having either the worker leave the mobile testing structure 100 or the patient leave their vehicle. The windows 113 may include gasket seals to maintain air pressure in the mobile testing center 113 when the worker is not serving a patient and the window 113 is closed.

The screen 113A covers a portion of the window 113 and provides additional physical protections to the worker in that it helps to maintain a safe distance between the worker and the patient but may be a mesh or other similar structure which also allows for good visual and audio communication between the patient and the worker. The screen 113A also prevents the patient from contaminating the workstation 116 of the worker by not allowing the patient physical access to the interior of the mobile testing structure 100. The screen 113A may not cover a portion of the window 113 such that the worker may extend an arm through the window 113 to serve patients on foot and in vehicles of all standard heights. The opposite side of the mobile testing structure 100 from that depicted in FIG. 2 may be substantially the same as the side depicted in FIG. 2, except it may not include an exterior door 112. The length L of the mobile testing center may be about 20 feet, however other lengths may be used as well.

The windows 113 may be capable of opening and the air control system 111 may be capable of maintaining a higher pressure in the interior of the mobile testing structure 100 compared to the air pressure of the exterior even when the windows 113 are open.

FIG. 3 shows an example back view of a mobile testing structure 100. The back of the mobile testing structure 100 may include the air control system 111. The mobile testing structure 100 may be made of a shipping container or other similar materials. Accordingly, in one example, the air control unit 111 may be mounted in a door of the shipping container. However, this is only one example embodiment; the air control system 111 may be anywhere on or in the mobile testing structure 100. The steel sides of the shipping container advantageously provide a strong structure that is secure and robust and provides physical protection for the workers inside the structure. An example height H of the mobile testing structure 100 may be 8 feet 6 inches, however other heights may be used as well.

FIG. 4 shows an example top view of an interior of a mobile testing structure 100. The mobile testing structure 100 may include air control system 111, exterior door 112, general purpose room 115, workstations 116, workstation walls 116A, workstation doors 116B, and air transfer ducting 117. Each room (workspaces 116 and general purpose room 115) may include an air outlet 118A, 118B, 118C, respectively, and at least one ultraviolet (“UV”) sanitation light 119. The mobile testing structure 100 may also include several sensors and controllers (not shown), such as air pressure sensors, temperature sensors, air contaminant sensors, door and window sensors, and control panels, which assist the air control system 111 in controlling the quality and pressure of the air in the mobile testing structure 100. The general purpose room 115 may be separate from the workstations 116. The workstation walls 116A and workstation doors 116B may separate the general purpose room and each of the workstations 116. The workstations 116 may be enclosed such that air cannot pass directly from a first workstation 116 to a second workstation 116.

In the interior of the mobile testing structure 100, the floor may be a level sheet or epoxy product. The ceiling may be a sheet product of ceiling tiles with a sheet face. The interior of the mobile testing structure 100, on the walls or ceiling, may include IC (insulation contact) rated LED (light emitting diode) lights in the visible spectrum (not shown) for normal use, as well as UV (ultraviolet) sanitation lights 119 which may be used when the mobile testing structure 100 is not occupied to sanitize, by generating UV light, the mobile testing structure 100 from any pathogens that might be in the air or on surfaces of the mobile testing structure 100. The mobile testing center 100 may also include insulation, such as closed cell foam or bar insulation to assist the air control system in controlling the temperature in the mobile testing structure 100. Electrical boxes (not shown) may also be included, which are sealed with gaskets and fire caulk to stop air transfer like the Hilti SF2. As an additional safety feature to protect from pathogens, workspace surfaces in the mobile testing structure 100 may be copper or copper coated.

The exterior door 112 may open inward to the general purpose room 115 and include a gasket seal around the exterior door 112. These features provide an advantage of having the exterior door 112 have a better seal to help maintain positive air pressure in the interior of the mobile testing structure 100. Advantageously, the exterior door 112 does not interfere with traffic when the exterior door 112 is open because it opens inwardly. The exterior door 112 may also include a panic bar (not shown) for increased security. The exterior door 112 may also include a door closer or spring-loaded hinges to help maintain positive air pressure in the interior of the mobile testing structure 100

The air control system 111 may control the air of the mobile testing structure 100. The air control system 111 may control temperature through heating and air conditioning. The air control system 111 may control air quality by filtering the air pumped into the mobile testing structure 100. For example, a High Efficiency Particulate Air (“HEPA”) filter may be used by the air control system to filter the air. Further, a UV sanitizer may be used to sterilize air pumped into the mobile testing structure 100. An advantage of filtering and/or sanitizing the air is maintaining a safer work environment for the workers in the mobile testing center.

The air control system 111 may control the interior air pressure of the mobile testing structure 100 to maintain a positive air pressure compared to the exterior air pressure. An advantage to having positive air pressure in the mobile testing structure 100 is that air is constantly flowing out of the mobile testing structure 100 so there is much less chance for contamination of the mobile testing structure 100 from airborne pathogens or contaminants coming from the exterior of the mobile testing structure 100. Because workers may be working for a significant amount of time with the window 113 open, the air pressure of the workstation 116 may be constantly monitored to ensure that a proper pressure differential is maintained. Accordingly, the air control system 111 is configured to filter air and control air pressure and temperature in the interior of the mobile testing structure 100.

The air control system in conjunction with the air transfer ducting 117 and air outlets 118A-C may be configured to maintain an air pressure in the general purpose room 115 greater than an air pressure in the workstations 116. The ducting 117 may either be flex or hard ducting with a non-operable air diffuser. The ducting 117 and air outlets 118A-C may include a two-stage damper that maintains the air pressure in the general purpose room 115 greater than an air pressure in the workstations 116. For example, each of the air outlets 118A-C may include a damper. Together the dampers may maintain the air pressure in the general purpose room 115 greater than an air pressure in the workstations 116. The two-stage damper may be balanced during manufacture of the mobile testing structure 100 or at a different time. In some embodiments, the dampers may be controllable to separately control the air supply to each of the workstations 116 and the general purpose room 115.

An advantage to having an air pressure in the general purpose room 115 higher than the workstations 116 is to maintain air flowing from the general purpose room 115 to the workstations 116 and thus not into the general purpose room 115 from the workstations 116. Thus, any contamination of one workstation 116 can be prevented from entering the general purpose room 115 or the other workstation 116.

The workstation doors 116B may provide access from the general purpose room 115 to the workstations 116. The workstation doors 116B may have gaskets to help maintain the pressure differential between the general purpose room 115 and the workspaces 116.

The general purpose room 115 may have any number of uses or configurations. For example, the general purpose room 115 may be used as a laboratory for testing samples obtained from patients through the window 113 in the workstation 116. As other examples, the general purpose room 115 may be a storage room for supplies or an administrative room. As shown in FIG. 4, the workstations 116 may take up about half of the space of the mobile testing structure. However, the size of the general purpose room 115 and the workstations 116 may be changed according to the intended use of the rooms.

The mobile testing structure 100 may include two workstations 116. The workstations 116 may be physically isolated by the workstation walls 116A and the interior doors 116B. The workstation walls 116A may be made with light gauge or wood stud framing with drywall or similar materials that may be fully sealed (additional tape or sealant may be used) to prevent air from passing form one workstation 116 to another. The workstation walls 116A and the interior doors 116B (i.e., interior separators) in the interior of the structure may separate the general purpose room 115 and the workstations 1116 from each other such that the air control system 111 is capable of separately controlling an air pressure in each of the workstations 116 and the general purpose room 115A.

An example width W of the mobile testing structure 100 is 8 feet, however other widths may be used as well. In general, the height H, length L, and width W of the mobile testing structure 100 should allow the mobile testing center 100 to be transported by semi-truck or other such vehicles.

FIG. 5 shows an example front view of a mobile testing structure 100. The mobile testing structure 100 may include front windows 114 for the workers to see the waiting line 300 or other objects. This is especially useful if the workers must operate a traffic light or the like.

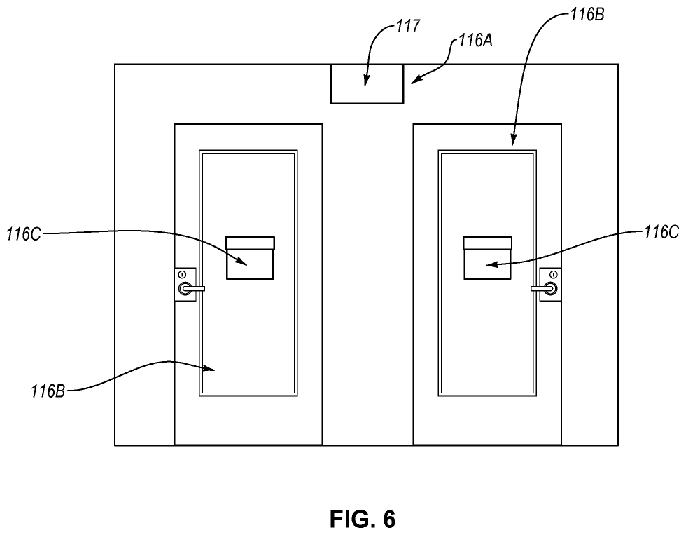

FIG. 6 shows workspace walls 116A and interior doors 116B with a small portal 116C included in the doors 116B. The small portals 116C may be placed in the workstation doors 116B to allow workers to pass supplies or medical tests between the workstation 116 and the general purpose room 115 without opening the interior doors 116B. The small portals 116C may be smaller than the doors 116B and may have a movable cover and a hole between the workstation and the general purpose room 115. The movable cover may cover the hole when the portal is not in use to reduce air flow from the general purpose room 115 to the workstation 116 and reduce the chance of contamination passing between the general purpose room 115 to the workstation 116.

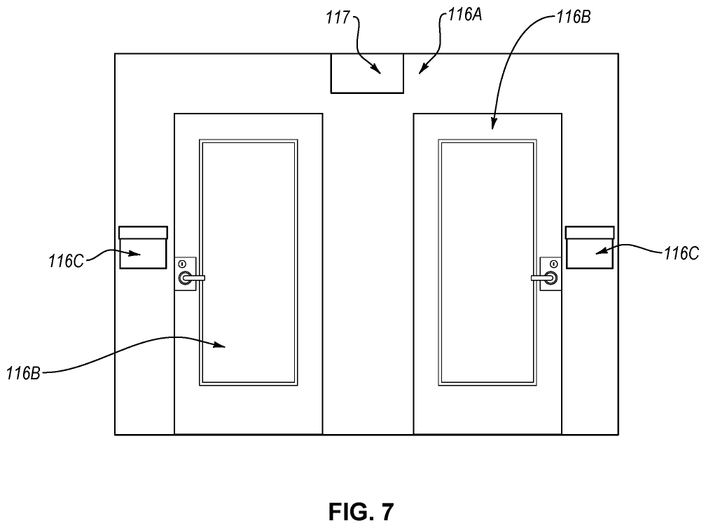

FIG. 7 shows workspace walls 116A and interior doors 116B with a small portal 116C included in the workspace walls 116A. The portal 116A may be placed in a workspace wall 116A between each workstation 116 and the general purpose room 115. The interior doors 116B may also include a door closer or spring-loaded hinges to help maintain air pressure differential in the interior of the mobile testing structure 100.

Many different embodiments of the inventive concepts have been shown. A person of ordinary skill in the art will appreciate that the features from different embodiments may be combined or replaced with other features from different embodiments.

As can be seen from the description above, the structure may provide significant advantages over the devices known in the art. The structure may provide significant advantages in cost and safety to workers over known structures or devices. The higher air pressure in the structure caused by the air control system may keep airborne pathogens out of the interior of the structure even when the windows are open. Having higher pressure in the general purpose room compared to the air pressure in the workstations also helps to prevent any contamination in the workstations from contaminating the other general purpose room or the other workstation(s).

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention.

The embodiments were chosen and described in order to best explain the principles of the invention and the practical application, and to enable others of ordinary skill in the art to understand the invention for various embodiments with various modifications as are suited to the particular use contemplated. The present invention according to one or more embodiments described in the present description may be practiced with modification and alteration within the spirit and scope of the appended claims. Thus, the description is to be regarded as illustrative instead of restrictive of the present invention.

What is claimed is:

1. A structure comprising:
a shell defining an interior of the structure;
at least one workstation in the interior of the structure;
at least one window in the shell, each of the at least one workstation including one of the at least one window communicating between the respective workstation and an exterior of the structure; and
an air control system configured to filter air and control air pressure in the interior of the structure.

2. The structure of claim 1, further comprising:
a room in the interior of the structure separate from the at least one workstation; and
interior separators in the interior of the structure which separate the room and each of the at least one workstation from each other, such that the air control system is capable of separately controlling an air pressure in each of the at least one workstation and the room.

3. The structure of claim 2, wherein the internal separators include a door for each of the at least one workstation for passage between the room and the respective at least one workstation.

4. The structure of claim 3, wherein the internal separators include a portal for each of the at least one workstation for passage between the room and the respective at least one workstation, wherein the portal is smaller than the door.

5. The structure of claim 2, wherein the air control system is configured to control air pressure within the room to be greater than air pressure within the at least one workstation.

6. The structure of claim 1, wherein the air control system is configured to control air pressure in the interior of the structure to be greater than an air pressure at the exterior of the structure.

7. The structure of claim 6, wherein each of the at least one window is capable of opening, and wherein the air control system is capable of maintaining a higher pressure in the interior of the structure compared to the air pressure of the exterior of the structure when the at least one window is open.

8. The structure of claim 1, wherein the air control system is configured to filter and pump in air from the exterior of the structure to the interior of the structure and is configured to control the air pressure within each of the at least one workstation by controlling how much air is pumped into each of the at least one workstation.

9. The structure of claim 1, wherein the at least one workstation includes a first workstation and a second workstation, and wherein the at least one workstation are enclosed such that air cannot pass directly from the first workstation to the second workstation.

10. The structure of claim 1, further comprising a plurality of UV sanitation lights configured to sanitize the interior of the structure.

11. A structure comprising:
a shell defining an interior of the structure;
at least one workstation in the interior of the structure;
a room in the interior of the structure separate from the workstation;
interior separators in the interior of the structure which separate the room and each of the at least one workstation from each other.
at least one window in the shell, each of the at least one workstation including one of the at least one window communicating between the respective workstation and an exterior of the structure; and
an air control system, ducting, and outlets configured to supply air to each of the at least one workstation and the room.

12. The structure of claim 11, wherein each of the at least one workstation and the room includes one of the outlets, wherein the outlets are separately controllable by the air controls system to control air supply to each of the at least one workstation and the room.

13. The structure of claim 11, wherein the internal separators include a door for each of the at least one workstation for passage between the room and the respective at least one workstation.

14. The structure of claim 11, wherein the internal separators include a portal for each of the at least one workstation for passage between the room and the respective at least one workstation, wherein the portal is smaller than the door.

15. The structure of claim 12, wherein the air control system is configured to control air pressure within the room to be greater than air pressure within the at least one workstation.

16. The structure of claim 11, wherein the air control system is configured to control air pressure in the interior of the structure to be greater than an air pressure at the exterior of the structure.

17. The structure of claim 16, wherein each of the at least one window is capable of opening, and wherein the air control system is capable of maintaining a higher pressure in the interior of the structure compared to the air pressure of the exterior of the structure when the at least one window is open.

18. The structure of claim 11, wherein the air control system is configured to filter and pump in air from the exterior of the structure to the interior of the structure and is configured to control the air pressure within each of the at least one workstation by controlling how much air is pumped into each of the at least one workstation.

19. The structure of claim 11, wherein the at least one workstation includes a first workstation and a second workstation, and wherein the at least one workstation are enclosed such that air cannot pass directly from the first workstation to the second workstation.

20. The structure of claim 11, further comprising a plurality of UV sanitation lights configured to sanitize the interior of the structure.
